(19) United States
(12) Patent Application Publication
(10) Pub. No.: US 2016/0136363 A1
(43) Pub. Date: May 19, 2016

(54) REPEATABLE INJECTION TARGETING SYSTEM

(71) Applicant: William T. MCCLELLAN, Morgantown, WV (US)

(72) Inventor: William T. MCCLELLAN, Morgantown, WV (US)

(21) Appl. No.: 14/542,958

(22) Filed: Nov. 17, 2014

(51) Int. Cl. A61M 5/42

(52) U.S. Cl. CPC A61M 5/427; A61M 2205/52; A61M 2205/505; A61M 2205/507

(57) ABSTRACT

Systems and method of targeted injection are provided. A method includes: capturing an image of a subject; displaying the image on a touch screen display; providing input to the touch screen display, wherein the input generates at least one mark superimposed on the image; saving the image and the at least one mark as a modified image; projecting the modified image onto the subject; and administering an injection to the subject where the at least one mark is projected on the subject.

### FIELD OF THE INVENTION

The invention generally relates to medical devices and procedures, and more particularly to systems and methods of administering repeated injections.

### BACKGROUND

Subcutaneous and intramuscular administration of a botulinum toxin is used for treating various diseases and for cosmetic applications. Typically, a syringe or a needleless device is used to inject the botulinum toxin to the dermal or subdermal target tissue. For some diseases, such as neuralgia, multiple injections of the botulinum toxin can be required over a relatively small area of the skin. Multiple injections are carried out to achieve a desired distribution and therapeutic diffusion of the botulinum toxin into the target area, as opposed to making only one or a few injections.

Injection therapy is commonly carried out over a number of discrete procedures that may be separated by weeks or months. In one exemplary scenario, a patient indicates that the previous injection treatment was highly satisfactory and requests that the provider “do what you did last time,” which essentially is a request to administer the injections of the current procedure in the exact same locations as the injections of the previous procedure. However, it is very difficult if not impossible for the provider to administer the injections in the exact same locations as they were administered in the previous office visit. This is because the provider is essentially going from memory as to where they administered the injections in the previous visit. As a result, it is nearly impossible to precisely repeat the same injections in the same locations from one injection treatment procedure to the next.

### SUMMARY

In a first aspect of the invention, there is a targeted injection therapy system comprising an imager that is configured to: capture an image of a subject; display the image; receive input from a user defining at least one mark superimposed on the image; save the image and the at least one mark as a modified image. The system also comprises a projector that is configured to receive the modified image from the imager and visually project the modified image onto the subject. The at least one mark comprises an injection guide.

In another aspect of the invention, there is a method of providing a targeted injection therapy system. The method includes providing a projector and a software program, wherein the software program is configured to be stored in a hardware memory of an imager and, when executed by a processor of the imager, cause the imager to: capture an image of a subject; display the image; receive input from a user defining at least one mark superimposed on the image; save the image and the at least one mark as a modified image. The projector is configured to receive the modified image from the imager and visually project the modified image onto the subject. The at least one mark comprises an injection guide.

In another aspect of the invention, there is a method of targeted injection, comprising: capturing an image of a subject; displaying the image on a touch screen display; providing input to the touch screen display, wherein the input generates at least one mark superimposed on the image; saving the image and the at least one mark as a modified image; projecting the modified image onto the subject; and administering an injection to the subject where the at least one mark is projected on the subject.

### BRIEF DESCRIPTION OF THE SEVERAL VIEWS OF THE DRAWINGS

The present invention is described in the detailed description which follows, in reference to the noted plurality of drawings by way of non-limiting examples of exemplary embodiments of the present invention.

FIGS. 1-3 show aspects of a system and method of use in accordance with aspects of the invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The invention generally relates to medical devices and procedures, and more particularly to systems and methods of administering repeated injections. A system according to aspects of the invention is structured and arranged to capture an image of a subject, receive modifications of the image including visual injection guides, and project the visual injection guides onto the subject. The provider administering an injection therapy to the subject may use the projected visual injection guides to precisely locate where to administer the injections on the subject. In embodiments, the system is configured to store the image and visual injection guides, and to project at least the visual injection guides onto the subject at a subsequent injection therapy procedure at a later date. In this manner, the system may be used to repeatedly guide the administration of injections on the subject at the same locations at different dates. Implementations of the invention thus facilitate providing reproducible results for the subject undergoing the injection therapy, which improves patient satisfaction with treatment and improves safety.

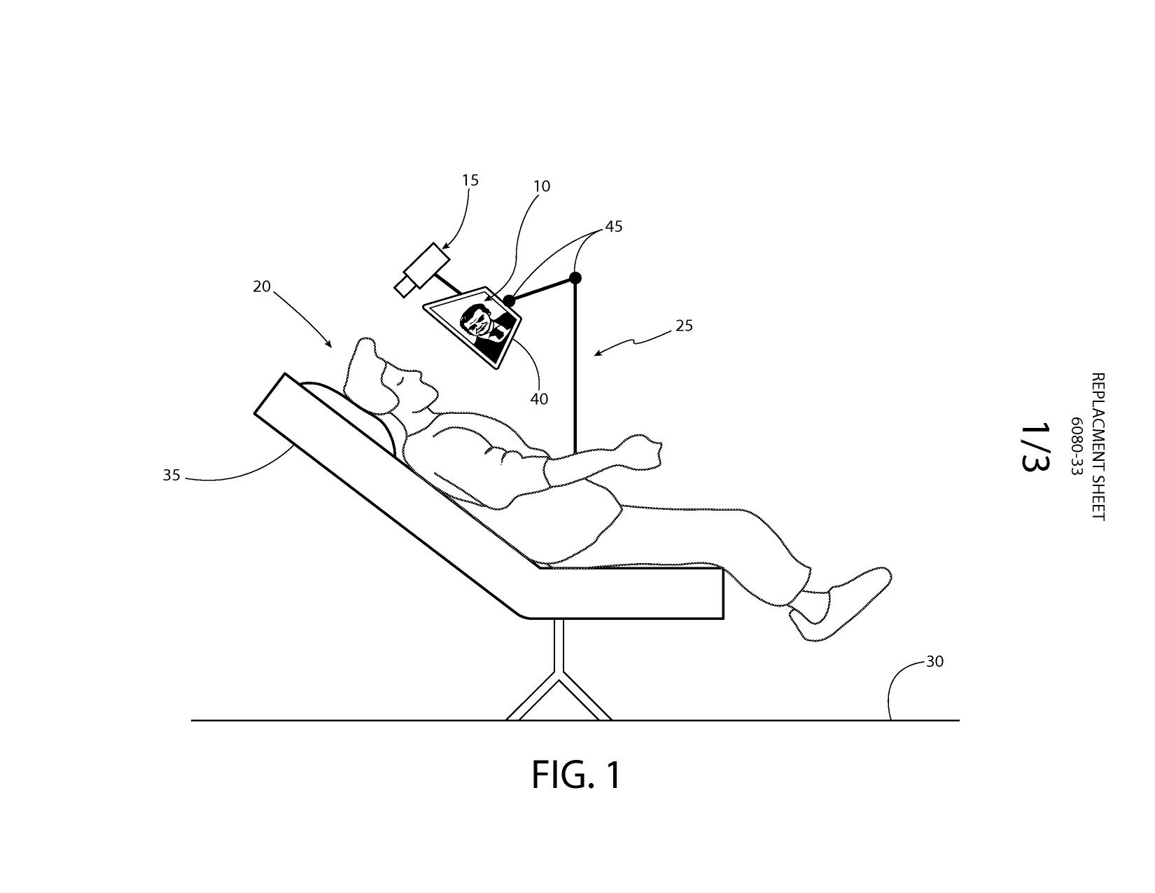

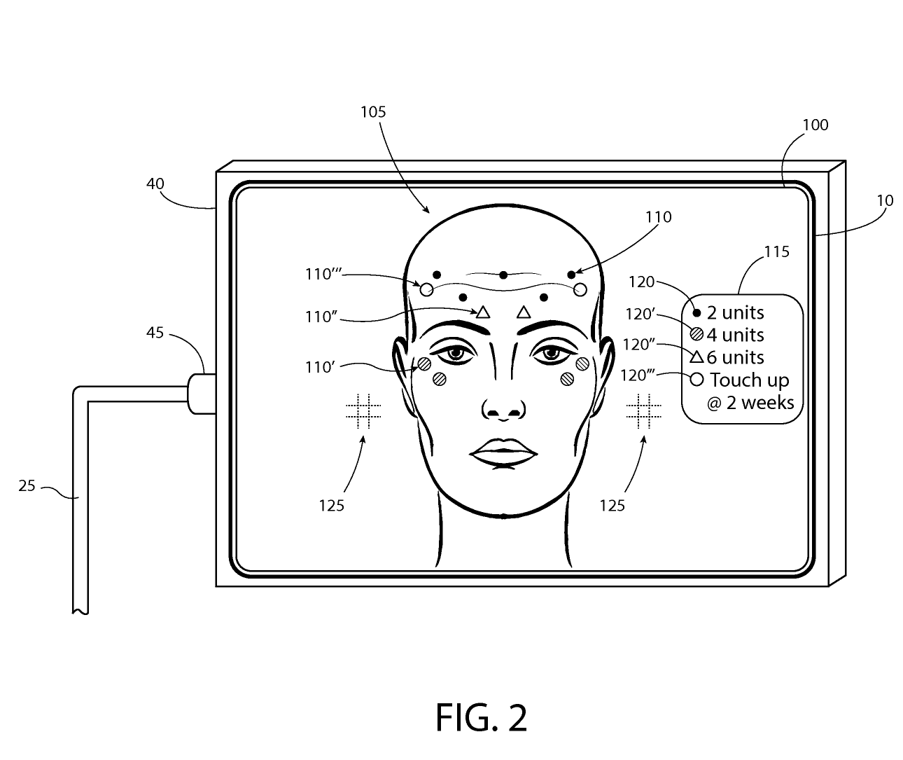

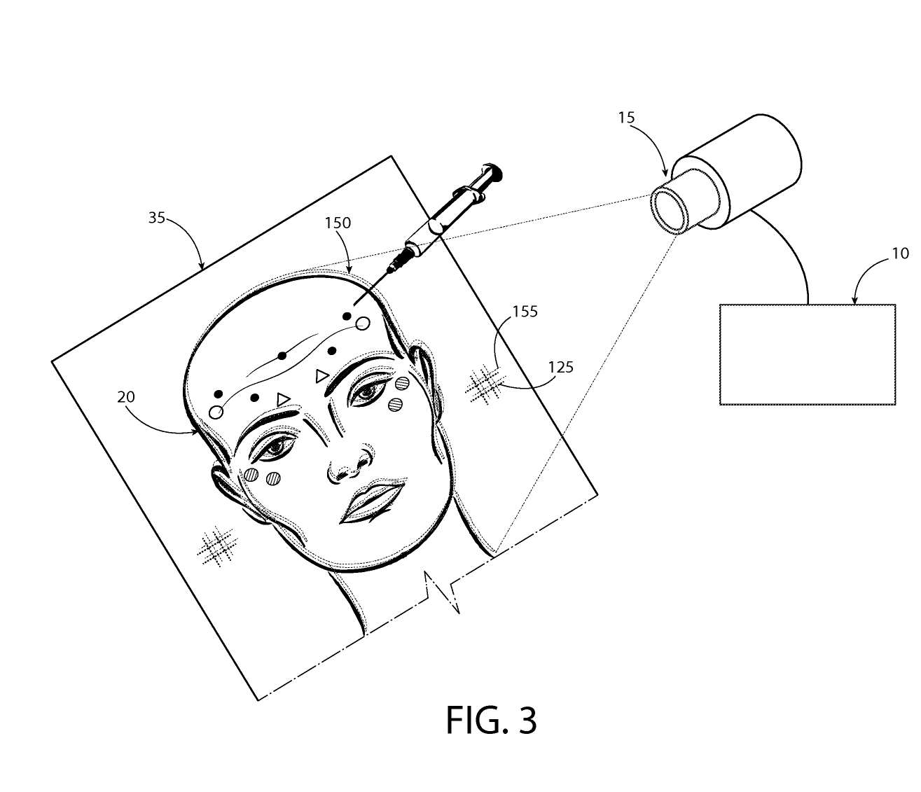

FIG. 1 shows an exemplary system in accordance with aspects of the invention comprising an imager 10 and a projector 15. The imager 10 is a device that is configured to capture an image of a subject 20, permit user-input modifications of the image, and save the modified image, e.g., as described in greater detail with respect to FIG. 2. The projector 15 is a device that is configured to communicate with the imager 10 and visually project the modified image, e.g., as described in greater detail with respect to FIG. 3. In embodiments, the imager 10 comprises a tablet computer, laptop computer, or smartphone that includes a camera, a processor, a memory, and a touch screen for accepting user input. A non-limiting example of one such computer is the iPad® available from Apple Inc. of Cupertino, Calif., although the invention may be implemented with an imager 10 comprising any suitable computer device that is configured to perform the functions described herein. The imager 10 may be programmed to perform one or more of the functions described herein, e.g., capture an image using a camera, display the image to a user, receive user inputs for modifying the image, store the modified image, and communicate the modified image to the projector 15.

As depicted in FIG. 1, an embodiment of the invention includes the imager 10 and projector 15 connected to a support 25 for stabilization. The support 25 may comprise a stand or frame that is connected to a floor 30, chair/table 35, ceiling, or other suitable structure. The support 25 may include a frame 40, clamp, or other structure for selectively connecting to and retaining the imager 10. The support 25 may also include at least one articulating joint 45 that is structured and arranged to permit the imager 10 to be moved relative to a stationary portion of the support 25 while the imager 10 is connected to the support 25. In this manner, when the imager 10 is connected to the support 25, the support 25 may be manipulated to move the imager 10 relative to a subject 20 to aim the camera of the imager 10 at the subject 20 for the purpose of capturing an image (e.g., a still photo or video) of the subject 20 with the imager 10.

Still referring to FIG. 1, the projector 15 may be integrated with the imager 10, connected directly to the imager 10, connected directly to the support 25, or connected to a separate support (not shown). In a particular embodiment, the projector 15 is connected to the frame 40 by a threaded connection, clamp, or similar mounting structure, e.g., in a manner similar to how a camera may be connected to a tripod by a threaded connection. The projector 15 may be any suitable image projector, i.e., an optical device that projects an image (or moving images) onto a surface. The projector 15 may utilize any suitable projection technology, such as DLP, CRT, LED, LCD, LCOS, FLCOS, etc. In embodiments, the projector 15 communicates via electronic signals with the imager 10 by a wired or wireless connection between the two devices. In this manner, the projector 15 is operatively connected to the imager 10 and configured to project an image provided by the imager 10.

FIG. 2 shows the imager 10 connected to the support 25 in which a display 100 of the imager 10 is displaying an image 105 of a subject (e.g., the subject 20 of FIG. 1). The display 100 may comprise, for example, a touch-screen display of an iPad® or similar tablet computer, laptop computer, smartphone, etc. In implementations, the imager 10 may be used to capture and display the image 105 in a conventional manner, e.g., using the camera of the iPad® to take a digital photograph and using the display screen of the iPad® to display the digital photograph.

Still referring to FIG. 2, in aspects of the invention the imager 10 is programmed to permit a user to modify the image 105 by adding one or more marks 110 superimposed on the image 105. The marks 110 may be any desired mark, including but limited to: dots or other geometric shapes, lines, alphabet characters, and numeral characters. The marks 110 may be created by the user using an input device of the imager 10, including but not limited to: touch-screen input with finger or stylus, mouse input, and trackball input. In a preferred embodiment, the imager 10 is programmed to create a mark 110 on the image 105 in a location where the user touches their finger to the display 100. The imager 10 is further programmed to save the image 105 and the marks 110 as a modified image, and to communicate the modified image to the projector 15 as described in greater detail herein.

With continued reference to FIG. 2, the imager 10 may be programmed to display a legend 115 that identifies different types of marks 110. For example, the legend 115 may include a first indicator 120 that corresponds in size/shape/color to a first type of mark 110, a second indicator 120′ that corresponds in size/shape/color to a second type of mark 110′, a third indicator 120″ that corresponds in size/shape/color to a third type of mark 110″, and a fourth indicator 120′″ that corresponds in size/shape/color to a fourth type of mark 110′″. The legend 115 may include text or other indicia associated with each respective indicator 120-120′″ that defines different types of injection therapy, e.g., two units, four units, six units, touch up in two weeks, etc. Implementations of the invention are not limited to four different types of marks and indicators as shown, but rather any suitable number may be used. Moreover, implementations of the invention are not limited to the specific text shown in the legend 115, but rather any desired text and/or indicia may be used.

In embodiments, the imager 10 is programmed to permit the user to define and selectively change the text that appears next to each indicator 120-120′″ in the legend 115, such that the user may customize the legend 115 to define a desired injection therapy. For example, the display 100 may show an options tab or button that the user selects to enter a temporary interface in which the user types the text associated with one or more of the indicators of the legend 115. The imager 10 may also be programmed to permit the user to define the number of types of marks. The imager 10 may also be programmed to permit the user to define at least one of a shape, a size, and a color of each type of mark. For example, the imager 10 may be programmed to display an interactive settings screen that permits the user to define these and other aspects. In embodiments, each one of the plurality of types of marks has a unique combination of color, size, and shape relative to other ones of the plurality of types of marks.

In additional embodiments, the legend 115 is interactively usable by the user to select which type of mark to place on the image 105. For example, the imager 10 may be programmed such that the user may select a respective type of mark 110-110′″ by touching (e.g., with their finger on the display 100) a predefined area of the legend 115 associated with a respective indicator 120-120′″. For example, the imager 10 may be programmed such that the user may select the first type of mark 110 by touching an area of the display 100 associated with the first indicator 120. After the user touches the area associated with the first indicator 120, the imager 10 superimposes a first type of mark 110 on the image 105 at each location where the user touches the display 100. The user may subsequently select the second type of mark 110′ by touching another portion of the display 100 associated with the second indicator 120′, after which the imager 10 superimposes the second type of mark 110 on the image 105 at each location where the user touches the display 100. In this manner, the user may select any one of the types of marks 110-110′″ via the legend 115 and then place an instance of the selected mark on the image 105 by touching the display at the desired location on the image 105.

Still referring to FIG. 2, the image 105 may include at least one alignment mark 125. In embodiments, the alignment marks 125 are used to align the projected modified image with the subject as described with respect to FIG. 3.

FIG. 3 shows the projector 15 projecting the modified image 150 onto the subject 20 in accordance with aspects of the invention. The modified image 150 is communicated to the projector 15 from the imager 10 and is visually projected by the projector 15. The modified image 150 includes the one or more marks 110 that are now projected into the face of the subject 20 by the projector 15. The provider may then use the one or more marks 110 as guides for determining where to administer injections into the subject 20.

With continued reference to FIG. 3, in embodiments the modified image 150 that is visually projected by the projector 15 includes the at least one alignment mark 125. The alignment marks 125 that are projected in the modified image 150 are captured as part of the image 105 when the imager takes a picture of the subject 20. For example, the alignment marks 125 may correspond to physical alignment marks 155 provided on a structure such as a chair/table 35. In this manner, when the subject 20 returns for a subsequent visit, the subject 20 may be seated on the same chair/table 35, and the projected image 150 may be aligned with the subject 20 by aligning the projected alignment marks 125 with the physical alignment marks 155. The alignment marks 125 may alternatively correspond to an anatomical feature of the subject 20, such as a scar, mole, etc. In this manner, when the subject 20 returns for a subsequent visit, the projected image 150 may be aligned with the subject 20 by aligning the projected alignment marks 125 with the anatomical feature on the subject 20.

A method according to aspects of the invention may include, for example: capturing an image of a subject using an imager; displaying the image on a visual display of the imager; receiving input from a user defining at least one mark superimposed on the image; saving the image and the at least one mark as a modified image; communicating the modified image to a projector; visually projecting the modified image onto the subject; and administering an injection to the subject at a location where the at least one mark of the modified image is projected onto the subject. The method may also include aligning the projected modified image with the subject, wherein the aligning includes aligning at least one alignment mark of the projected modified image with a physical alignment mark. The receiving input from the user defining the at least one mark may include receiving a selection of one type of mark from a plurality of different types of marks. The method may include displaying the plurality of different types of marks on the visual display of the imager, and the receiving the selection may include receiving an input associated with one of the displayed plurality of different types of marks.

Additional aspects of the invention include a method comprising providing a system comprising the imager 10 and the projector 15, in which the imager 10 is configured to perform one or more of the functions described herein (e.g., capturing an image of a subject; displaying the image on a visual display; receiving input from a user defining at least one mark superimposed on the image; saving the image and the at least one mark as a modified image; communicating the modified image to a projector).

Additional aspects of the invention include a method comprising providing a software program that is configured to be stored in hardware memory of an imager 10 and that, when executed by a processor of the imager 10, causes the imager 10 to perform one or more of the functions described herein (e.g., capturing an image of a subject; displaying the image on a visual display; receiving input from a user defining at least one mark superimposed on the image; saving the image and the at least one mark as a modified image; communicating the modified image to a projector). The software program may be, for example, an app that is stored and executed on an iPad® to perform the functions described herein. The method may include: storing a copy of the app at a remote computer device (e.g., a server computer device comprising at least a processor and a hardware memory, such as that used in an app store); receiving, at the remote computer device, a request for the app from a requesting device (e.g., from the imager 10 via network communication); and transmitting, by the remote computer device, a copy of the app to the requesting device.

Additional aspects of the invention include a method of providing a targeted injection system comprising: providing the projector 15 and the software program that is configured to be stored in hardware memory of an imager 10 and that, when executed by a processor of the imager 10, causes the imager 10 to perform one or more of the functions described herein (e.g., capturing an image of a subject; displaying the image on a visual display; receiving input from a user defining at least one mark superimposed on the image; saving the image and the at least one mark as a modified image; communicating the modified image to a projector). In this implementation, the user already owns the imager, e.g., an iPad®. A vendor sells the projector to be operatively connected to the imager, and a copy of the software program to be installed on the imager.

Even further aspects of the invention include providing instructions for using the imager 10 in the manner described herein. The instructions may be at least one of printed and video.

Implementations of the invention may be used to provide accuracy and repeatability in administering injections to a subject. Aspects may be used to provide targeted treatment of: brow lines, glabella furrows, crows feet, nasojugal folds, nasolabial folds, marionette lines, chin line, and platysmal bands. Implementations of the invention are not limited to use with the face of a subject, and instead may be used on any part of a subject. Aspects described herein permit the provider to reproduce injections over multiple visits and/or alter injection habits in order to maximize the subject results. Aspects described herein also provide the ability to add alterations of treatments, such as touch ups, bruises, complications, etc., to the picture of the subject. As described herein, implementations of the invention provide the ability to access a saved image and treatment guide that was given to the subject at a prior date, and to re-project that same image and treatment guide onto the subject to administer injections in the exact same locations as the previous visit. In embodiments, the imager is configured to save plural images of a single subject, e.g., in a subject file, and to permit the user to search and view the stored images to identify good and/or bad locations related to results. Aspects described herein permit the user to save a record of injection treatment locations and amounts (e.g., dosages) using the marks that are superimposed on the image of the subject.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A targeted injection therapy system, comprising:
an imager that is configured to: capture an image of a subject; display the image; receive input from a user defining at least one mark superimposed on the image; save the image and the at least one mark as a modified image; and
a projector that is configured to receive the modified image from the imager and visually project the modified image onto the subject,
wherein the at least one mark comprises an injection guide.

2. The system of claim 1, wherein the imager comprises:
a camera;
a touch screen display;
a hardware memory; and
a processor.

3. The system of claim 2, wherein:
the imager captures the image of the subject using the camera;
the imager displays the image using the touch screen display; and
the imager receives the input from the user defining the at least one mark via the touch screen display while the image is displayed on the touch screen display.

4. The system of claim 3, wherein the imager displays the at least one mark superimposed on the image in real time as the user is providing the input via the touch screen display.

5. The system of claim 3, wherein:
the imager displays a legend comprising a plurality of types of marks;
the imager receives input from the user comprising a selection of one of the plurality of types of marks.

6. The system of claim 5, wherein each one of the plurality of types of marks has a unique combination of color, size, and shape relative to other ones of the plurality of types of marks.

7. The system of claim 6, wherein the imager is configured to receive input from the user defining at least one of the color, size, and shape of each of the plurality of types of marks.

8. The system of claim 5, wherein, for each one of the plurality of types of marks, the imager displays respective text that indicates dosage and/or time.

9. The system of claim 8, wherein the imager is configured to receive input from the user defining the respective text.

10. The system of claim 5, wherein the plurality of types of marks comprise:
a first mark associated with a first injection dose;
a second mark associated with a second injection dose; and
a third mark associated with a third injection dose.

11. The system of claim 1, wherein the imager provides the modified image to the projector by wired or wireless communication.

12. The system of claim 1, further comprising a support structure, wherein the imager and the projector are connected to the support structure.

13. The system of claim 12, wherein the support structure comprises at least one articulating joint that is structured and arranged to permit the imager to be moved relative to first portion of the support structure while the imager is connected to the support structure.

14. The system of claim 1, wherein:
the image comprises an alignment mark; and
the modified image comprises the alignment mark.

15. The system of claim 14, wherein the alignment mark corresponds to a physical alignment mark provided on a structure adjacent to the subject.

16. A method of providing a targeted injection therapy system, comprising:
providing a projector and a software program, wherein:
the software program is configured to be stored in a hardware memory of an imager and, when executed by a processor of the imager, cause the imager to: capture an image of a subject; display the image; receive input from a user defining at least one mark superimposed on the image; save the image and the at least one mark as a modified image;
the projector is configured to receive the modified image from the imager and visually project the modified image onto the subject; and
the at least one mark comprises an injection guide.

17. A method of targeted injection, comprising:
capturing an image of a subject;
displaying the image on a touch screen display;
providing input to the touch screen display, wherein the input generates at least one mark superimposed on the image;
saving the image and the at least one mark as a modified image;
projecting the modified image onto the subject; and
administering an injection to the subject where the at least one mark is projected on the subject.

18. The method of claim 17, further comprising:
prior to the capturing the image, positioning a subject adjacent to a structure that has a physical alignment mark such that the image includes an alignment mark that corresponds to the physical alignment mark; and
prior to the administering the injection, positioning the subject adjacent to a structure that has a physical alignment mark and aligning the alignment mark in the projected modified image with the physical alignment mark.

19. The method of claim 17, wherein:
the capturing, the displaying, and the providing input are performed at a first time; and
the projecting and the administering the injection are performed at a second time later than the first time.

20. The method of claim 17, further comprising selecting the at least one mark from a legend comprising a plurality of types of marks.
